(19) United States
(12) Patent Application Publication
(10) Pub. No.: US 2025/0032688 A1
(43) Pub. Date: Jan. 30, 2025

(54) PROCESS OF DISINFECTING A FLUID LINE SYSTEM OF A MEDICAL APPARATUS AND MEDICAL APPARATUS

(71) Applicant: B. Braun Avitum AG, Melsungen (DE)

(72) Inventors: Nino Lieb, Melsungen (DE); Philipp Odernheimer, Melsungen (DE); Jonas Giesa, Kassel (DE)

(21) Appl. No.: 18/782,312

(22) Filed: Jul. 24, 2024

(30) Foreign Application Priority Data
Jul. 25, 2023 (DE) 10 2023 119 706.1

(51) Int. Cl. A61M 1/16; A61L 2/04; A61L 2/24; A61M 1/14

(52) U.S. Cl. CPC A61M 1/1686; A61L 2/04; A61L 2/24; A61M 1/154; A61M 1/169; A61L 2202/14; A61L 2202/24; A61M 2205/3368; A61M 2205/3606; A61M 2209/10

(57) ABSTRACT

An apparatus and process are used to disinfect a fluid line system of a medical apparatus. A heating coefficient, passive cooling coefficient, and ready-to-use time are considered when defining a target curve of fluid line system temperature. The heating coefficient indicates a time-dependent rise of system temperature when the fluid line system is heated. The passive cooling coefficient indicates a time-dependent decrease of system temperature when the fluid line system is passively cooled. The ready-to-use time is a time at which system temperature must be less than or equal to a maximum ready-to-use temperature, after the last time the temperature falls below a minimum disinfection temperature, so that the medical apparatus is ready-to-use after disinfection. Before a minimum value of target lethality is reached, and before the last time the temperature falls below the minimum disinfection temperature, cooling occurs by passive cooling by stopping or interrupting heating.

### CROSS-REFERENCE TO RELATED APPLICATION

This application claims priority under 35 U.S.C. § 119 to German Application No. 10 2023 119 706.1, filed on Jul. 25, 2023, the content of which is incorporated by reference herein in its entirety.

### FIELD

The present disclosure relates to a process of disinfecting (i.e., of reducing germs by a factor of at least 10-5) of a fluid line system of a medical apparatus as well as to a medical apparatus.

### BACKGROUND

In known apparatuses for disinfection, disinfection processes are performed time- and temperature-controlled or via an A0 value calculation.

In time- and temperature-controlled disinfection processes, a target value is set for a disinfection temperature. After the disinfection temperature is reached, a timer starts to record a holding and safety time. When the required disinfection temperature is maintained over the entire holding and safety time, the disinfection is successful. Then a cooling phase takes place.

In disinfection processes with A0 value calculation, a target value is set for the A0 value. After a defined temperature is reached during the heating phase, the time and the temperature values are taken into account for the A0 value calculation. When the A0 value is reached, the cooling phase takes place.

The known processes are not optimal in terms of energy, because more energy is supplied to the system than is required for disinfection. Temperature and time values or, resp., the corresponding A0 value are not taken into account during the cooling phase. In apparatuses having an active cooling, the time at which a required target temperature has to be reached is not evaluated, which results in an increased need of cooling water.

### SUMMARY

Therefore, it is the object of the present disclosure to facilitate a disinfection of a fluid line system of a medical apparatus which is more efficient in terms of heat to be generated and/or quantity of disinfectant to be used.

A process according to the disclosure is configured to disinfect a fluid line system of a medical apparatus, specifically a blood treatment apparatus, preferably a dialysis machine, or a reverse-osmosis system. “To disinfect” means in particular to achieve germ reduction by a factor between including 10-5 and excluding 10-6.

The process comprises the following steps of:
measuring a fluid line system temperature in a predetermined temperature measuring section within the fluid line system, specifically by means of at least one temperature sensor, preferably by means of plural temperature sensors distributed over the fluid line system, wherein for monitoring and/or carrying out a disinfection preferably a measuring value of the temperature sensor which measures the lowest temperatures as compared to the remaining temperature sensors can be used as fluid line system temperature, and for monitoring and/or carrying out a cooling preferably a measuring value of the temperature sensor which measures the highest temperatures as compared to the remaining temperature sensors is used as fluid line system temperature, or wherein, alternatively, the measuring values of the temperature sensors can be averaged, for example,
defining a target curve of the fluid line system temperature by means of a predetermined minimum value of a target lethality of germs potentially present in the temperature measuring section, wherein the target lethality is calculated by means of integration of the target curve of the fluid line system temperature over time from the first time a minimum disinfection temperature is reached from which germs potentially present in the temperature measuring section can be killed until the last time the temperature falls below the minimum disinfection temperature,
heating the fluid line system by means of a disinfectant fluid in (time or heating time) periods of the target curve of the fluid line system temperature in which the fluid line system temperature is to be increased or kept constant, and
cooling the fluid line system in (time or cooling time) periods of the target curve of the fluid line system temperature in which the fluid line system temperature is to be lowered.

An actual lethality is calculated particularly corresponding to the target lethality. That is, an actual curve of the fluid line system temperature is integrated over time from the first time the minimum disinfection temperature is reached until the last time the temperature falls below the minimum disinfection temperature. When calculating the target lethality and, correspondingly, when calculating the actual lethality, particularly periods in which the measured fluid line system temperature is less than the minimum disinfection temperature are not considered and, resp., not integrated.

According to the disclosure, when defining the target curve of the fluid line system temperature, a heating coefficient, a passive cooling coefficient and a standby time are taken into account.

The heating coefficient indicates a time-dependent increase in the fluid line system temperature when the fluid line system is heated. The passive cooling coefficient indicates a time-dependent decrease of the fluid line system temperature when the fluid line system is passively cooled. The ready-to-use time is a time at which the fluid line system temperature, after the last time the temperature falls below the minimum disinfection temperature, must be less than or equal to a maximum ready-to-use temperature so that the apparatus is ready for use again after disinfection.

According to the disclosure, before the predetermined minimum value of the target lethality is reached and before a value of the actual lethality corresponds to the predetermined minimum value of the target lethality and before the last time the temperature falls below the minimum disinfection temperature, cooling of the fluid line system takes place at least temporarily by means of the passive cooling by solely stopping or interrupting heating.

The fact that, when calculating the actual lethality, also periods after stopping or interrupting heating are considered, allows a use time of a heater or a heater and a duration of a disinfection process to be reduced while the predetermined minimum value of the target lethality is kept constant.

According to an aspect of the disclosure, the heating coefficient and/or the passive cooling coefficient can be defined by means of at least one test and/or by means of a simulation before defining the target curve of the fluid line system temperature, particularly before or after measuring the fluid line system temperature in the predetermined temperature measuring section. The heating coefficient can be detected and, resp., established using measurement data in particular during or at the beginning of the process while a heater is operated. The passive cooling coefficient can be detected or established using measurement data in particular during or at the beginning of the process by temporarily switching off the heater. The heating coefficient and/or the passive cooling coefficient can be adapted stepwise specifically by means of interlinked devices which analyze measurement data via cloud and/or edge computing. The heating coefficient and/or the passive cooling coefficient can be calculated in particular by thermodynamic equations, preferably iteratively, by means of a control device according to the disclosure or a calculation device. The simulation or a simulated model of the fluid line system for determining the heating coefficient and/or the passive cooling coefficient can be created by the control device according to the disclosure or the calculation device, in particular on the basis of fluid data (for example a specific heat capacity, a total volume), a pipe geometry (length, diameter and/or material thickness), insulation (of the lines), one or more heat transfer coefficients of the line/system material, an outside temperature and/or an effective heating power in the fluid line system.

If the heating coefficient and/or the passive cooling coefficient is defined before the target curve of the fluid line system temperature is defined, heating and/or cooling phases can be advantageously considered during disinfection.

According to an aspect of the disclosure, the heating coefficient and/or the passive cooling coefficient can be monitored while the process is carried out and, if the heating coefficient and/or the passive cooling coefficient change(s), the step of defining the target curve of the fluid line system temperature can be carried out repeatedly. In other words, the heating coefficient and/or the passive cooling coefficient can be re-calculated based on measurement data while the process is carried out.

If the heating coefficient and/or the passive cooling coefficient is/are monitored and adjusted, if necessary, the accuracy can be improved when calculating the actual lethality.

According to an aspect of the disclosure, when the target curve of the fluid line system temperature is defined, an active cooling coefficient indicative of a time-dependent decrease of the fluid line system temperature during an active cooling of the fluid line system can be considered, and the cooling of the fluid line system can take place temporarily by means of the active cooling by discharging hot disinfection fluid and/or supplying a coolant. The disinfection fluid can be mixed specifically with chemicals. The coolant can particularly be of the type of a fluid, such as a process water, which is used during regular operation of the medical apparatus. The active cooling coefficient can be calculated in particular by thermodynamic equations, preferably iteratively, by means of the control device according to the disclosure or the calculation device. The simulation or a simulated model of the fluid line system for determining the active cooling coefficient can be created by the control device according to the disclosure or the calculation device, in particular on the basis of fluid data (for example a specific heat capacity, a total volume), a pipe geometry (length, diameter and/or material thickness), insulation (of the lines), one or more heat transfer coefficients of the line/system material, an outside temperature and/or an effective heating power in the fluid line system.

If an active cooling coefficient is considered when the target curve is defined, phases in which the fluid line system is actively cooled can advantageously be considered during disinfection.

According to an aspect of the disclosure, the active cooling coefficient can be defined before the target curve of the fluid line system temperature is defined, specifically before or after the fluid line system temperature is measured in the predetermined temperature measuring section, by means of at least one test and/or by means of a simulation. The active cooling coefficient can be detected and established, resp., on the basis of measurement data particularly during or at the beginning of the process by temporarily switching off the heater and simultaneously discharging hot disinfection fluid and/or supplying a coolant.

If the heating coefficient is defined before the target curve of the fluid line system temperature is defined, active cooling phases can advantageously be considered during disinfection.

According to an aspect of the disclosure, the active cooling coefficient can be monitored while the process is carried out, and the step of defining the target curve of the fluid line system temperature can be carried out repeatedly, if the active cooling coefficient changes. In other words, the active cooling coefficient can be re-calculated on the basis of measurement data while the process is carried out.

If the active cooling coefficient is monitored and adjusted, if necessary, the accuracy in defining the target curve can be improved in terms of reaching the ready-to-use time.

According to an aspect of the disclosure, by means of the predetermined target lethality, the ready-to-use time, the passive cooling coefficient and the active cooling coefficient, a last possible active cooling starting time can be calculated according to which the maximum ready-to-use temperature can be reached only by means of active cooling and until which the fluid line system is cooled exclusively by means of the passive cooling. The passive cooling can be carried out specifically largely by free convection.

According to an aspect of the disclosure, a safety factor can be included when the predetermined minimum value of the target lethality is defined.

If the predetermined minimum value of the target lethality is defined to be larger than theoretically required, a probability of unintentionally inadequate disinfection can be reduced.

According to an aspect of the disclosure, the target curve of the fluid line system temperature can be defined so that an average level of the fluid line system temperature between the first time the minimum disinfection temperature is reached and the last time the temperature falls below the minimum disinfection temperature is above the minimum disinfection temperature.

If the average level of the fluid line system temperature is defined to be above the minimum disinfection temperature, a probability of unintentionally inadequate heating can be reduced.

According to an aspect of the disclosure, the average level of the fluid line system temperature between the first time the minimum disinfection temperature is reached and the last time the temperature falls below the minimum disinfection temperature can be lowered in the direction of the minimum disinfection temperature, if it is detected that, after the last time the temperature falls below the minimum disinfection temperature, the fluid line system temperature can be lowered to the maximum ready-to-use temperature without the active cooling before the ready-to-use time.

If the average level of the fluid line system temperature is lowered in the direction of the minimum disinfection temperature as needed, the resource efficiency of the disinfection can be increased.

Moreover, the disclosure relates to a medical apparatus, in particular a blood treatment apparatus, preferably a dialysis machine, or a reverse-osmosis system, comprising a fluid line system, at least one temperature sensor that is designed to be capable of measuring a fluid line system temperature in a predetermined temperature measuring section within the fluid line system, an inlet for introducing a disinfectant fluid and/or a coolant into the fluid line system, an outlet for discharging the disinfectant fluid and/or the coolant from the fluid line system, a heater for heating the disinfectant fluid and a control device for controlling the inlet, the outlet and the heater in response to the fluid line system temperature measured by the at least one temperature sensor.

According to the disclosure, the control device is designed to carry out a process according to the disclosure in response to a request.

### BRIEF DESCRIPTION OF THE DRAWINGS

In the following, the present disclosure will be described in detail on the basis of preferred embodiments with reference to the attached drawings, wherein:

FIG. 1 shows a schematic view of a medical apparatus according to the disclosure as set forth in a first embodiment;

FIG. 2 shows a schematic view of a medical apparatus according to a second embodiment;

FIG. 3 shows a schematic temperature-time diagram of a process according to the disclosure;

FIG. 4 shows a diagram in which the temperature and the A0 value are shown over time during a process according to the disclosure until a predetermined minimum value of a lethality is reached;

FIG. 5 shows a diagram corresponding to FIG. 4 concerning a process according to the disclosure in which an increased predetermined minimum value of the lethality is reached; and

FIG. 6 shows diagrams in which the temperature, the A0 value and a system state of a medical apparatus are shown during a process according to the disclosure.

### DETAILED DESCRIPTION

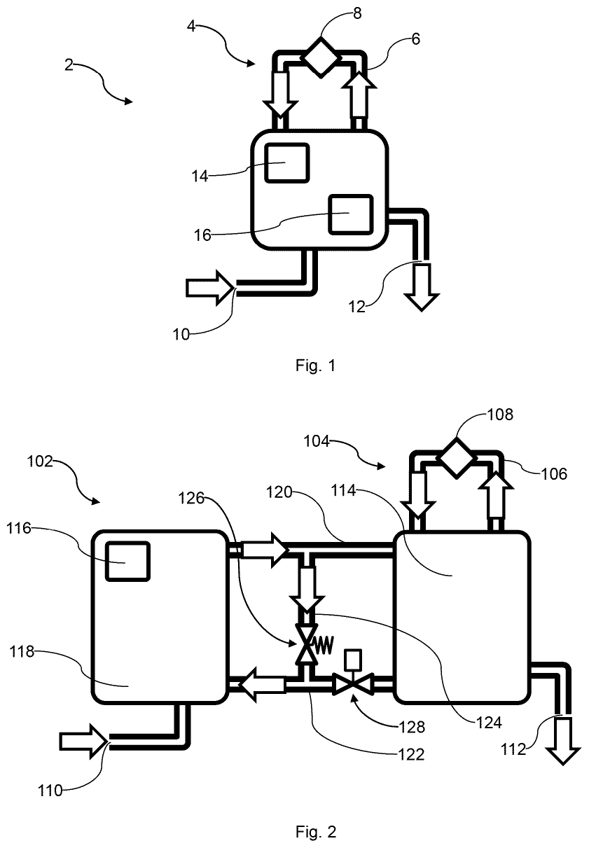

FIG. 1 illustrates a schematic view of a medical apparatus 2 according to the disclosure as set forth in a first embodiment. The apparatus is in the form of a reverse-osmosis system and includes a fluid line system 4. The fluid line system 4 is equipped with a ring line 6 on which a temperature sensor 8 is provided. The temperature sensor 8 is designed to be capable of measuring a fluid line system temperature according to the disclosure. As an alternative, plural temperature sensors distributed over the fluid line system 4 could be provided, wherein for monitoring and/or carrying out a disinfection a measuring value of the temperature sensor which measures the lowest temperatures as compared to the remaining temperature sensors is used as fluid line system temperature, and for monitoring and/or carrying out a cooling a measuring value of the temperature sensor which measures the highest temperatures as compared to the remaining temperature sensors is used as fluid line system temperature. In addition, temperature sensors can be present in the medical apparatus 2, particularly outside the fluid line system 4, which can be equally used for calculation, in particular if it cannot be excluded that the hottest or coolest point is located outside the fluid line system 4.

The apparatus 2 includes an inlet 10 through which a disinfectant fluid and/or cooling water can be introduced into the fluid line system 4 in the apparatus 2.

The apparatus 2 further includes an outlet 12 through which disinfectant fluid and/or coolant can be discharged from the apparatus 2.

The apparatus 2 is equipped with a heater 14 which is designed to be capable of warming up or heating disinfectant fluid provided in the apparatus 2 and, resp., in the fluid line system 4.

The apparatus 2 includes a control device 16 which is designed to be capable of controlling the inlet 10, the outlet 12 and/or the heater 14 in parallel or serially.

In order to disinfect the apparatus 2 and, resp., the fluid line system 4, process water which is heated in the apparatus 2 using the heater 14 is introduced through the inlet 10. The heated water is distributed as disinfectant fluid in the fluid line system 4 and, resp., in the ring line 6. In order to deliver fluid in the apparatus 2 and, resp., in the fluid line system 4, the apparatus 2 may include a pump (not shown).

The disinfection is controlled by the control device 16 by means of calculating a lethality of germs potentially present in the fluid line system 4 and, resp., in the ring line 6.

As a characteristic for lethality, the A0 value which is calculated by the following formula according to ISO 15883 is used:

$${A}_{0} = \sum 1 {0}^{\frac{[ T ( t ) - 80 ° C . ]}{z}} \cdot Δ t$$

“z” is the z value for the definition of which ISO 11139 can be resorted to, for example. For the A0 value “z=10° C.” is applicable.

“T(t)” is the temperature of the disinfectant fluid in ° C. in response to the time “t”.

“Δt” is the selected time interval in seconds.

As soon as a predetermined minimum value of the A0 value was reached, the apparatus 2 and, resp., the control device 16 stops the thermal disinfection and changes to a cooling phase by discharging hot water from the outlet 12 and replacing it with colder process input water.

FIG. 2 illustrates a schematic view of a medical apparatus 102 according to a second embodiment. The apparatus 102 according to the second embodiment differs from the apparatus 2 according to the first embodiment in that in the latter embodiment the heater 14 has an integral design, whereas a heater 114 according to the second embodiment is designed separately from a reverse-osmosis unit 118. The heater 114 is connected to the reverse-osmosis unit 118 via a cooling fluid line 120 and a hot fluid line 122.

Via the cooling fluid line 120, fluid can be fed from the reverse-osmosis unit 118 to the heater 114. Via the hot fluid line 122, fluid can be fed from the heater 114 to the reverse-osmosis unit 118. The cooling fluid line 120 and the hot fluid line 122 are connected to each other via a return line 124 which laterally branches off a section of the cooling fluid line 120 and laterally branches into a section of the hot fluid line 122. An overflow valve 126 is provided in the return line 124. A shift valve 128 is provided upstream of the branching of the return line 124 in the hot fluid line 122.

Corresponding to the apparatus 2 according to the first embodiment, the apparatus 102 according to the second embodiment includes a fluid line system 104 having a ring line 106. The ring line 106 is provided with a temperature sensor 108 so that the fluid line system temperature of a fluid present in the fluid line system 104 and, resp., in the ring line 106 can be measured. Alternatively, plural temperature sensors distributed over the fluid line system 104 could be provided, wherein for monitoring and/or carrying out a disinfection a measuring value of the temperature sensor which measures the lowest temperatures as compared to the remaining temperature sensors is used as fluid line system temperature, and for monitoring and/or carrying out a cooling a measuring value of the temperature sensor which measures the highest temperatures as compared to the remaining temperature sensors is used as fluid line system temperature.

The apparatus 102 includes an inlet 110 at the reverse-osmosis unit 118 via which a disinfectant fluid and/or cooling water can be introduced into the fluid line system 104 in the apparatus 102.

The apparatus 102 moreover includes an outlet 112 at the heater 114 via which disinfectant fluid and/or coolant can be discharged from the apparatus 102.

Just as the integral heater 14 according to the first embodiment, the separately designed heater 114 according to the second embodiment is designed to be capable of warming up or heating disinfectant fluid present in the apparatus 102 and, resp., in the fluid line system 104.

In the reverse-osmosis unit 118 and/or the heater 114, the apparatus 102 includes a control device 116 which is designed to be capable of controlling the inlet 110, the outlet 112 and/or the heater 114 in parallel or serially.

For a heat disinfection of the ring line 106 without heat disinfection of the reverse-osmosis unit 118, the heater 114 is switched on and the heated water is fed into the ring line 106. The shift valve 128 is closed to keep the hot water inside the heater 114 and the ring line 106. Should the heater 114 require fresh water, this will be re-supplied by the reverse-osmosis unit 118. The excess water flows back into the reverse-osmosis unit 118 via the overflow valve 126 in the return line 124.

For a heat disinfection of the ring line 106 and the reverse-osmosis unit 118, the heater 114 is switched on and the heated water is fed into the ring line 106. The shift valve 128 is opened to feed the hot water equally into the reverse-osmosis unit 118. The overflow valve 126 is adjusted so that a permeate of the reverse-osmosis unit 118 has to flow through the heater 114. “Permeate” is understood to be specifically water which was separated, by means of a semipermeable membrane of the reverse-osmosis unit 118, from components which were dissolved in the water before. The permeate is particularly suited to be used for the preparation of a dialysis fluid without any further treatment.

The disinfection of the apparatus 102 according to the second embodiment is also controlled using a calculation of the A0 value. That is, as soon as a predetermined minimum value of the A0 value has been reached, the apparatus 102 stops the thermal disinfection and changes to the cooling phase by discharging hot water through the outlet 112 and replacing it with colder process inlet water.

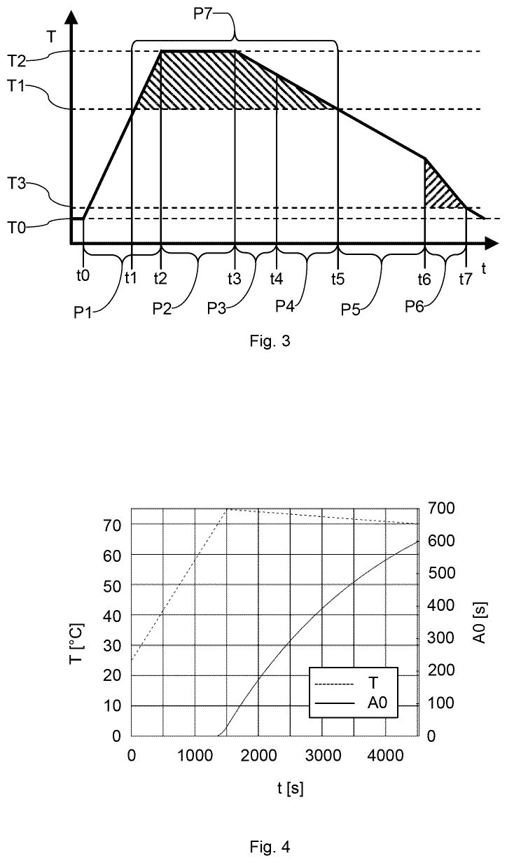

FIG. 3 illustrates a schematic temperature-time diagram of a process according to the disclosure for disinfection of the apparatus 2 and, resp., 102 and a target curve of the fluid line system temperature.

At a time t0 it is started to heat the fluid line system 4 and, resp., 104 which at the beginning of the disinfection process has the ambient temperature TO.

At a time t1, the fluid line temperature reaches a minimum disinfection temperature T1 from which germs are killed in the fluid line system 4 and, resp., 104.

The fluid line temperature is further increased after the time t1, until a maximum heating temperature T2 is reached at a time t2.

From the time t2, the fluid line temperature is kept constant at the value of the maximum heating temperature T2, until the heater 14 and, resp., 114 is switched off at a time t3.

From the time t3, the fluid line system 4 and, resp., 104 cools passively, i.e., without any action of a cooling device.

At a time t4, the A0 value, i.e., the surface between the target curve of the fluid line temperature and the isotherm of the minimum disinfection temperature T1 between t1 and t4 hatched in FIG. 3, reaches a value from which the fluid line system 4 and, resp., 104 can be considered to be disinfected. In order to take any discrepancies during disinfection into account, the target curve of the fluid line temperature is defined so that an A0 value (corresponding to a predetermined minimum value of a target lethality according to the disclosure) reached when the last time the temperature falls below the minimum disinfection temperature T1 at a time t5 is larger than the theoretically required A0 value (at the time t4).

From the time t5, the fluid line system 4 and, resp., 104 continues cooling passively before an active cooling is switched on at a time t6 (“switch on active cooling” means that hot disinfectant fluid is discharged from the fluid line system 4 and, resp., 104 through the outlet 12 and, resp., 112 and cold process inlet water is introduced into the fluid line system 4 and, resp., 104 through the inlet 10 and, resp., 110.

From the time t6, the fluid line system 4 and, resp., 104 is actively cooled so that at a ready-to-use time t7 the fluid line system temperature reaches a maximum ready-to-use temperature T3. The maximum standby temperature T3 in the present example is higher than the ambient temperature TO. As an alternative, also the ambient temperature TO can be defined as the maximum ready-to-use temperature T3.

The period from including the time t0 to excluding the time t2 is a heating phase P1. The period from including the time t1 to excluding the time t3 is a holding phase P2. The period from including the time t3 to excluding the time t4 is a passive cooling disinfection phase P3. The period from including the time t4 to excluding the time t5 is a safety phase P4. The period from including the time t5 to excluding the time t6 is a passive cooling phase P5. The period from including the time t6 to excluding the time t7 is an active cooling phase P6. The period from including the time t1 to excluding the time t5 is a disinfection phase P7.

The gradient of the target curve of the fluid line system temperature in the heating phase P1 corresponds to a heating coefficient according to the disclosure. The gradient of the target curve of the fluid line system temperature in the passive cooling disinfection phase P3, the safety phase P4 and the passive cooling phase P5 corresponds to a passive cooling coefficient according to the disclosure. The gradient of the target curve of the fluid line system temperature in the active cooling phase P6 corresponds to an active cooling coefficient according to the disclosure.

FIG. 4 shows a diagram in which the fluid line system temperature and the A0 value is illustrated over time during a process according to the disclosure until a predetermined minimum value of a lethality is reached. In the example shown in FIG. 4, the starting or ambient temperature TO is equal to 25° C., the predetermined minimum value of the target lethality at the time t5=4500 s is equal to 600, the heating coefficient is 2° C./min and the passive cooling coefficient is 6° C./h.

It can be detected that the active heat cleaning takes only 25.04 minutes (1502.3 seconds) and then changes to the standby mode. By the temperature of 75.04° C. reached in this way and the assumed cooling, an A0 value of 600 is reached. In the example shown in FIG. 4 no holding phase is provided.

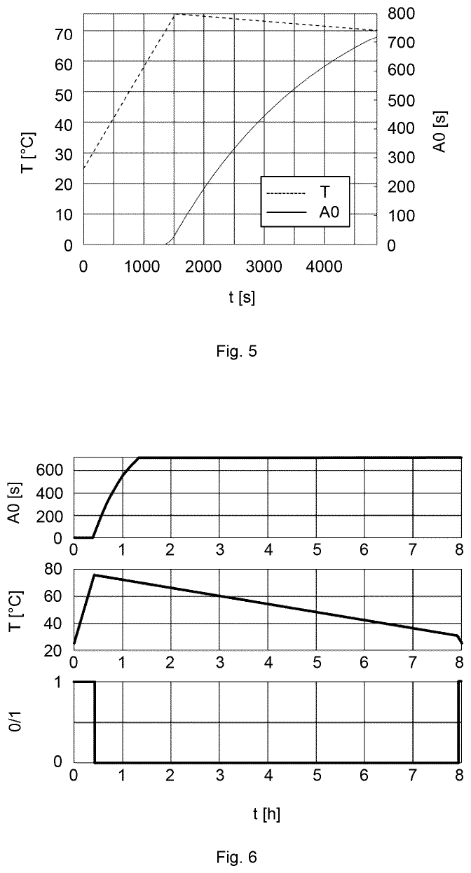

FIG. 5 shows a diagram corresponding to FIG. 4 concerning a process according to the disclosure in which an increased predetermined minimum value of the lethality is reached. In order to reach the predetermined minimum value of the lethality increased as compared to the example shown in FIG. 4 of 720 at the time t5=5000 s, a higher maximum heating temperature T2 is provided. Since the predetermined minimum value of the lethality of 720 is reached at a later time than in the example shown in FIG. 4, the use of an active cooling is rather necessary in the example shown in FIG. 5.

FIG. 6 shows diagrams from which the fluid line system temperature, the lethality and, resp., the A0 value as well as a system state of a medical apparatus 2 and, resp., 102 can be seen even after the predetermined minimum value of the lethality is reached.

As in the examples illustrated in FIGS. 4 and 5, no holding phases are provided. That is to say, as soon as the fluid line system temperature reaches the maximum heating temperature T2 or, resp., as soon as the target curve of the fluid line system temperature allows to reach the disinfection target, the heater 14 and, resp., 114 is switched off in the shown examples.

The predetermined minimum value of the lethality is reached after approx. 25 minutes (i.e., t5=25 min). To ensure that the apparatus 2 and, resp., 102 is ready at a predetermined ready-to-use time, the apparatus is actively cooled from the latest possible time t6. In the diagram at the bottom of FIG. 6, the activation of the apparatus 2 and, resp., 102 is shown. That is to say, it is shown in the bottom diagram whether or not the heater 14 and, resp., 114, pumps and valves of the apparatus 2 and, resp., 102 are switched off. Preferably, the apparatus 2 and, resp., 102 switches off all energy consumers (except the control unit) to save energy during passive cooling.

In a further advantageous configuration, the pumps/valves can be switched at short notice during the passive disinfection/cooling operation to circulate the water. The aim is to ensure an as homogenous temperature distribution in the system as possible.

In the example illustrated in FIG. 6, the apparatus 2 and, resp., 102 only needs to be actively cooled for 190 seconds from about 30° C. to 25° C. before the ready-to-use time. Consequently, by taking into account the ready-to-use time, it is possible to save plenty of water and/or energy as compared to the known disinfection processes.

### LIST OF REFERENCE SIGNS

2; 102 apparatus
4; 104 fluid line system
6; 106 ring line
8; 108 temperature sensor
10; 110 inlet
12; 112 outlet
14; 114 heater
16; 116 control device
118 reverse-osmosis unit
120 cooling fluid line
122 hot fluid line
124 return line
126 overflow valve
128 shift valve
t0 time of start of heating phase
t1 time of start of disinfection process
t2 time of reaching the maximum heating temperature
t3 time of switching off a heater
t4 time of reaching a theoretically sufficient predetermined minimum value of a lethality
t5 time of reaching a minimum value of a lethality predetermined with a safety factor
t6 time of switching on an active cooling
t7 ready-to-use time
T0 starting or ambient temperature
T1 minimum disinfection temperature
T2 maximum heating temperature
T3 maximum ready-to-use temperature
P1 heating phase
P2 holding phase
P3 passive cooling disinfection phase
P4 safety phase
P5 passive cooling phase
P6 active cooling phase
P7 disinfection phase

1. A process of disinfecting a fluid line system of a medical apparatus, the process comprising the steps of:
measuring a fluid line system temperature in a predetermined temperature measuring section inside the fluid line system;
defining a target curve of the fluid line system temperature by a predetermined minimum value of a target lethality of germs potentially present in the predetermined temperature measuring section, wherein the target lethality is calculated by integration of the target curve of the fluid line system temperature over time from a first time a minimum disinfection temperature sufficient to kill germs potentially present in the predetermined temperature measuring section is reached until a last time the fluid line system temperature falls below the minimum disinfection temperature;
heating the fluid line system with a disinfection fluid in sections of the target curve of the fluid line system temperature in which the fluid line system temperature is to be increased or kept constant; and
cooling the fluid line system in sections of the target curve of the fluid line system temperature in which the fluid line system temperature is to be lowered,
wherein,
when the target curve of the fluid line system temperature is defined, a heating coefficient indicative of a time-dependent rise of the fluid line system temperature when the fluid line system is heated, a passive cooling coefficient indicative of a time-dependent decrease of the fluid line system temperature when the fluid line system is passively cooled, and a ready-to-use time at which the fluid line system temperature must be less than or equal to a maximum ready-to-use temperature after the last time the fluid line system temperature falls below the minimum disinfection temperature so that the medical apparatus is ready-to-use after disinfection, are taken into account and
wherein, before the predetermined minimum value of the target lethality is reached and before the last time the fluid line system temperature falls below the minimum disinfection temperature, cooling of the fluid line system takes place at least temporarily by passive cooling solely by stopping or interrupting heating.

2. The process according to claim 1, wherein the heating coefficient and/or the passive cooling coefficient is/are defined, before the target curve of the fluid line system temperature is defined, by at least one test and/or by a simulation.

3. The process according to claim 1, wherein the heating coefficient and/or the passive cooling coefficient is/are defined, before or after the fluid line system temperature is measured in the predetermined temperature measuring section, by at least one test and/or by a simulation.

4. The process according to claim 1, wherein the heating coefficient and/or the passive cooling coefficient is/are monitored while the process is carried out and, when the heating coefficient and/or the passive cooling coefficient is/are changed, the step of defining the target curve of the fluid line system temperature is carried out repeatedly.

5. The process according to claim 1, wherein, when the target curve of the fluid line system temperature is defined, an active cooling coefficient indicative of a time-dependent decrease of the fluid line system temperature when the fluid line system is actively cooled, is taken into account and the fluid line system is cooled temporarily by active cooling by discharging hot disinfectant fluid and/or supplying a coolant.

6. The process according to claim 5, wherein the active cooling coefficient is defined before the target curve of the fluid line system temperature is defined by at least one test and/or by means of a simulation.

7. The process according to claim 5, wherein the active cooling coefficient is defined before or after the fluid line system temperature is measured in the predetermined temperature measuring section by at least one test and/or by means of a simulation.

8. The process according to claim 5, wherein the active cooling coefficient is monitored while the process is carried out and, when the active cooling coefficient changes, the step of defining the target curve of the fluid line system temperature is carried out repeatedly.

9. The process according to claim 5, wherein:
a last possible active cooling starting time is calculated by the target lethality, the ready-to-use time, the passive cooling coefficient and the active cooling coefficient,
the maximum ready-to-use temperature is reachable only by active cooling after the last possible active cooling starting time, and
the fluid line system is cooled exclusively by passive cooling until the last possible active cooling starting time.

10. The process according to claim 1, wherein a safety factor is included when defining the predetermined minimum value of the target lethality.

11. The process according to claim 1, wherein the target curve of the fluid line system temperature is defined so that an average level of the fluid line system temperature between the first time the minimum disinfection temperature is reached and the last time the fluid line system temperature falls below the minimum disinfection temperature lies above the minimum disinfection temperature.

12. The process according to claim 11, wherein the average level of the fluid line system temperature between the first time the minimum disinfection temperature is reached and the last time the fluid line system temperature falls below the minimum disinfection temperature is lowered in a direction of the minimum disinfection temperature, when the fluid line system temperature is capable of being lowered, after the last time the fluid line system temperature falls below the minimum disinfection temperature, without active cooling before the ready-to-use time to the maximum ready-to-use temperature.

13. A medical apparatus comprising:
a fluid line system,
at least one temperature sensor which is designed to be capable of measuring a fluid line system temperature in a predetermined temperature measuring section inside the fluid line system,
an inlet for introducing a disinfectant fluid and/or a coolant into the fluid line system,
an outlet for discharging the disinfectant fluid and/or the coolant from the fluid line system,
a heater for heating the disinfectant fluid, and
a control device for controlling the inlet, the outlet and the heater in response to the fluid line system temperature measured by the at least one temperature sensor,
wherein
the control device is designed to carry out the process according to claim 1 upon request.

14. The medical apparatus according to claim 13, where the medical apparatus is a blood treatment apparatus.

15. The medical apparatus according to claim 14, where the medical apparatus is a dialysis machine or a reverse-osmosis system.
